(19) United States
(12) Patent Application Publication
(10) Pub. No.: US 2006/0146984 A1
(43) Pub. Date: Jul. 6, 2006

(54) Method for determining at least one scaling factor for measured values of a computed tomography unit

(76) Inventors: Herbert Bruder, Hoechstadt (DE); Johannes Ebersberser, Erlangen (DE); Rainer Raupach, Adelsdorf (DE)

Correspondence Address: HARNESS, DICKEY & PIERCE, P.L.C., P.O.BOX 8910, RESTON, VA, 20195, US

(21) Appl. No.: 11/313,732

(22) Filed: Dec. 22, 2005

(30) Foreign Application Priority Data
Dec. 27, 2004 (DE) 10 2004 062 857.2

(51) Int. Cl. A61B 6/00; G01N 23/00; G21K 1/12; H05G 1/60

(52) U.S. Cl. 378/9

(57) ABSTRACT

A method is disclosed for determining at least one scaling factor for measured values obtained with the aid of a computed tomography unit. The computed tomography unit includes at least two recording systems that can rotate about a common rotation axis. Each of the systems includes an X-ray source and a detector having detector elements for detecting X-radiation emanating from the X-ray source. To reduce artifacts when use is made of measured values of the two recording systems in the reconstruction of an image, a scaling factor is determined for the measured values of the first or of the second recording system on the basis of measured values that originate from projections recorded from an object with the aid of the two recording systems. Each of the two recording systems is used to record at least one projection at at least substantially the same projection angle, whose measured values are compared with one another.

The present application hereby claims priority under 35 U.S.C. §119 on German patent application number DE 10 2004 062 857.2 filed Dec. 27, 2004, the entire contents of which is hereby incorporated herein by reference.

### FIELD

The invention generally relates to a method for determining at least one scaling factor for measured values obtained with the aid of a computed tomography unit. The computed tomography unit may, for example, include at least two recording systems that can rotate about a common rotation axis and of which each includes an X-ray source and a detector, having detector elements, for detecting X-radiation emanating from the X-ray source.

### BACKGROUND

A known computed tomography unit is disclosed, for example, in DE 103 02 565 A1. By comparison with a computed tomography unit having only one recording system, the advantage of a computed tomography unit having two or more recording systems resides in an increased data recording rate that leads to a shorter recording time, and in an increased temporal resolution. A shortened recording time is advantageous because it reduces or even minimizes movement artifacts in the reconstructed image caused, for example, by voluntary or involuntary movements of a recorded object.

This is important above all in the medical field, when a relatively large volume, for example, of the heart, is recorded, particularly during a spiral scan. An increased temporal resolution is required, for example, to display movement sequences, because then the data used to reconstruct an image must be recorded in the shortest possible time.

However, it has emerged that whenever such a computed tomography unit is used to reconstruct images of an object that are based on measured values of the two recording systems, artifacts occur in the images. The cause of the artifacts is the various scalings of the measured values obtained with the aid of the two recording systems. The various scalings of the measured values result from mutually independent settings of the two recording systems before the commissioning of the computed tomography unit for object measurements.

Specifically for the detector of a recording system, it is necessary before the commissioning to carry out various steps for calibration, normalization and correction of measured values, and/or to record various correction tables and store them for later signal processing, in order to be able to reconstruct high quality images from the measured values of said detector. Offset correction tables, channel error correction tables, radiation hardening correction tables and water scaling factors may be named here by way of example. The determination of the correction tables is necessary because the detector elements that form the detector differ slightly from one another in their measurement response because of tolerances, although the detector elements of a detector are already preselected such that they exhibit at least substantially the same response.

An X-ray computed tomography unit with a detector having detector elements and in the case of which the detector elements are calibrated by comparing detector element output values, a correction factor being determined, is disclosed, for example, in EP 0 089 096 B1.

The detector elements of different detectors are not tuned to one another as a rule. Consequently, the response of two different detectors also does not correspond as a rule. The scalings based on the correction tables for the measured values obtained with the two detectors are determined independently of one another such that there is thus no tuning of the detectors.

High quality images are obtained by reconstructing images from measured values of each detector per se. However, if the measured values of the two detectors are brought together in the case of a computed tomography unit of the type mentioned at the beginning, the various scalings give rise to data discontinuities that cause the artifacts, already mentioned above, in the reconstructed image.

### SUMMARY

It is an object of at least one embodiment of the invention to specify a method for determining at least one scaling factor for a computed tomography unit such that the occurrence of artifacts is at least reduced when an image is reconstructed by using the measured values of the two recording systems.

According to at least one embodiment of the invention, an object may be achieved by a method for determining at least one scaling factor for measured values obtained with the aid of a computed tomography unit, which computed tomography unit has at least two recording systems that can rotate about a common rotation axis and of which each includes an X-ray source and a detector, having detector elements, for detecting X-radiation emanating from the X-ray source. In order to reduce the occurrence of artifacts when use is made of measured values of the two recording systems in the reconstruction of an image, it is provided according to at least one embodiment of the invention to determine a scaling factor for the measured values of the first or of the second recording system. The scaling factor is determined in this case from measured values that originate from projections recorded from an object with the aid of the two recording systems, at least one projection at substantially the same projection angle being recorded with the aid of each of the two recording systems.

Consequently, according to at least one embodiment of the invention, the measured values obtained with the aid of the detectors of the two recording systems can be compared with one another, and it is possible, preferably in a global fashion, to determine an associated scaling factor for one of the two recording systems. Measured values, based on the projections, of corresponding detector elements of the two detectors are compared, as a rule, or mean values determined for each detector, from the measured values, based on the projections, of the detector elements of a detector. By taking account of the scaling factor determined, it is possible in this way to bring together the measured values recorded with the aid of the two recording systems after scaling as a function of the image to be reconstructed, and to reconstruct an image of a recorded object in which the artifacts otherwise occurring are at least reduced and, in some circumstances, even completely avoided.

Should artifacts appear, nevertheless, instead of a globally determined associated scaling factor, it could be required to determine a number of scaling factors for various sections of a detector and/or for various groups of detector elements of a detector such that a corresponding scaling factor is assigned to each relevant detector section of one of the two detectors of the two recording systems.

Embodiments of the invention provide that when determining the scaling factor measured values of corresponding detector elements of the two recording systems, which are located substantially at the same or a corresponding position in space during recording of the respective projections, are compared with one another in pairs. The measured values of corresponding detector elements are preferably divided. Because of the noise of the measured values, according to one variant of an embodiment of the invention, averaging is carried out over the divided measured values. Since, as mentioned at the beginning, the detector elements of a detector are selected in such a way that they behave substantially identically, it is possible in this way to determine an associated scaling factor for each of the two, or else both recording systems.

Another embodiment of the invention provides that averaging is carried out in each case over the measured values of the projection that is recorded with the aid of each of the two recording systems at substantially the same projection angle. The mean values determined are subsequently divided in order to determine the scaling factor.

According to one variant of an embodiment of the invention, the scaling factor is determined by using projections of the two recording systems recorded at various projection angles. Thus, a number of projection pairs are available for determining the scaling factor, one projection of a projection pair being recorded with the aid of the first recording system, and the other projection of the projection pair being recorded with the aid of the second recording system, respectively at substantially the same projection angle.

Another variant of an embodiment of the invention provides that use is made when determining the scaling factor of a number of projections that are obtained in one or in a number of different segments of a scan. Thus, in this case it is only projection pairs of a specific sector of a scan, that is to say projections that have been recorded for specific projection directions, and are used to determine the scaling factor, and this can be advantageous wherever it is easier to evaluate for the determination of the scaling factor the projections of the object that is being used to determine the scaling factor which are recorded in a specific sector.

According to a further variant of an embodiment of the invention, in order to determine the scaling factor in the case when a number of projection pairs of the two recording systems are used to determine the scaling factor, averaging is carried out over measured values of a detector element of each detector that originate from projections obtained at various projection angles, and the averaged measured values of corresponding detector elements of the two detectors are divided. If the aim is global determination of an associated scaling factor, averaging is then carried out again over these determined values in order to obtain the scaling factor.

At least one embodiment of the invention provides that the determination of the scaling factor is performed in the course of the water value scaling of the two recording systems before an object measurement. Here, the water value scaling is the last step in the generation, addressed at the beginning, of correction tables and correction values for the CT raw data processing. In the case of the water value scaling, a water scaling factor is determined for one recording system, and is intended to be used to multiply normalized and corrected measured values already calibrated in some other way, so that the CT values in an image of a centric circular water disk that has been reconstructed from the measured values of the recording system are on average at 0 HU (Hounsfield unit). This water value scaling is preferably carried out for both recording systems such that the two recording systems can be used independently of one another for imaging.

According to another variant of at least one embodiment of the invention, the scaling factor is determined during an object measurement with the aid of the two recording systems. It is thus also possible in the course of an object measurement for corresponding projections, that is to say a projection pair, to be recorded at at least substantially the same projection angle, and for the measured values of the two projections to be compared with one another in order to determine the scaling factor. This mode of procedure is suggested, in particular, for checking the scaling factor during operation of the computed tomography unit. Specifically, drifting of the measured values can set in as time progresses, for example owing to ageing phenomena of the detector elements; these can be countered by redetermining the scaling factor.

According to a particular example embodiment of the invention, a number of scaling factors are determined as a function of the slice thickness of the X-ray beam, which is shaped as a rule with the aid of diaphragms, and can lead from the X-ray source, and the energy of the X-radiation, and are stored for later signal processing in a memory of the computed tomography unit. As a rule, the X-ray source is an X-ray tube, and so the scaling factors can be determined as a function of the high voltages applied to the X-ray tube. The dependence of the scaling factor on the slice thickness is explained by the different scattered beam acceptance of a detector. Thus, the hardening correction corrects not only radiation hardening effects, but also nonlinearities owing to scattered beam capture from the water phantom used as a rule for the calibration, for which reason there are slight variations in the effective radiation attenuation values which are a function of slice thickness.

### BRIEF DESCRIPTION OF THE DRAWINGS

An example embodiment of the invention is illustrated in the attached schematic figures, in which:

FIG. 1 shows a computed tomography unit having two recording systems, in an overview representation,

FIG. 2 shows a sectional illustration of the two recording systems of the computed tomography unit from FIG. 1, and

FIG. 3 shows a sectional illustration of the two recording systems of the computed tomography unit from FIG. 1, in another position than in FIG. 2.

### DETAILED DESCRIPTION OF THE EXAMPLE EMBODIMENTS

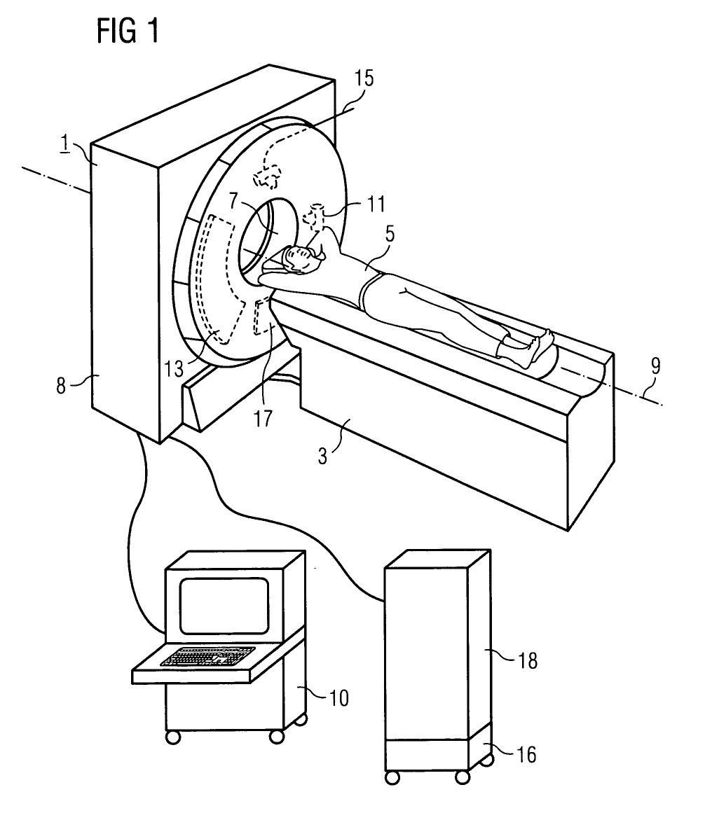

FIG. 1 shows in an overview representation a computed tomography unit 1 with a support device 3, having a moveable table plate, for holding and supporting an object. Supported in FIG. 1 on the support device 3 is a patient 5 who can be introduced, by way of the moveable table plate, into a patient opening 7 in a housing 8, the examination or scanning region, of the computed tomography unit 1.

In its housing 8, the computed tomography unit 1 has two recording systems that can rotate about a common axis of rotation 9. The first recording system includes an X-ray source in the form of an X-ray tube 11, and an X-ray detector 13, a multirow one in the present case, opposite the X-ray tube 11. The second recording system, which in the case of the present example embodiment is arranged in the same plane of rotation as the first recording system, likewise includes an X-ray source in the form of an X-ray tube 15, and an X-ray detector 17, a multirow one in the present case, opposite the X-ray tube 15.

Owing to the fact that the two recording systems are arranged in a common plane, the X-ray beams emanating from the two X-ray sources 11 and 15 are also located, at least substantially, in the same plane. The two recording systems are, moreover, arranged in a way not shown in more detail in FIG. 1 on a common rotary carriage that can rotate about the axis of rotation 9.

In order to examine the patient 5, the latter is brought into the patient opening 7 in the housing 8 by adjusting the table plate such that X-ray projections of the patent 5 can be obtained in a so-called scan with the aid of one or both recording systems from various projection directions. The projections are preferably obtained in a spiral scan in the case of which the table plate is moved into or through the patient opening 7 in the housing 8 during the rotation of the two recording systems. A control and image computer 18 of the computed tomography unit 1 can reconstruct a 2- or 3-dimensional image of the recorded body region of the patient 5 in a known way from the X-ray projections recorded at various projection angles. The projections obtained with the aid of the two recording systems can be used here independently of one another to reconstruct an image.

However, the measured values originating from the projections of the two recording systems are also mixed with one another, depending on the application present, in order by applying an image reconstruction algorithm known per se to derive therefrom an image of the recorded body region of the patient 5, which can be a tomogram or a volume image. In order to operate the computed tomography unit 1, moreover, a separate operating unit 10 is provided in the case of the present example embodiment.

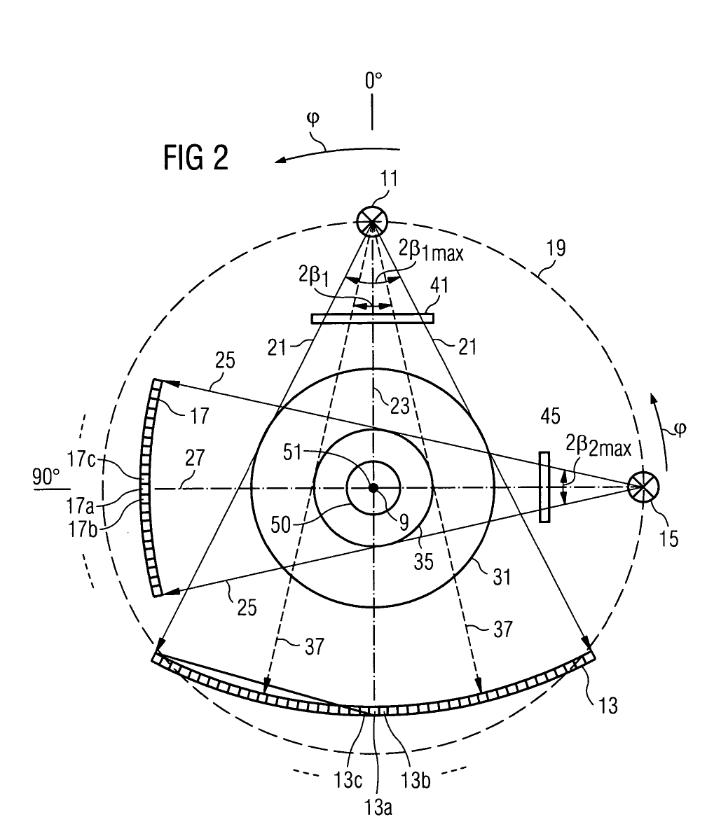

The arrangement of the two recording systems is illustrated once again more accurately in FIG. 2. It is to be seen from FIG. 2 that the X-ray tube 11 of the first recording system is assigned a diaphragm 41 with which it is possible to vary not only the aperture angle, as illustrated in FIG. 2, but also the thickness, to be measured in the direction of the axis of rotation 9, of the fan-shaped or pyramid-shaped X-ray beam, the slice thickness of an object to be transradiated thereby being fixed. Shown in FIG. 2 for the first recording system is the maximum fan aperture angle 2β₁ₘₐₓ, for which the X-ray beam with the middle ray 23 and the edge rays 21 illuminates the entire detector 13. A measuring field 31 can be scanned upon rotation of the recording system in the φ-direction, during which the X-ray tube 11 moves on the circulating track 19. In this case, the detector 13 of the present exemplary embodiment also has a number of rows of detector elements. However, only one detector row with detector elements 13a, 13b, . . . , etc. is shown in FIG. 2. If the aperture angle is reduced to 2β₁, only a part of the detector 13 is illuminated, and the reduced measuring field 35 results.

The second recording system is arranged in relation to the first recording system in a fashion offset by 90° about the axis of rotation 9, and has substantially the same design as the first recording system. The X-ray tube 15 is assigned a diaphragm 45 with the aid of which it is likewise possible to set the thickness of the X-ray beam emanating from the X-ray tube 15, as well as of the aperture angle. By contrast with the first recording system, the second recording system, however, has a smaller detector 17. Given a maximum aperture angle 2β₂ₘₐₓ, the detector 17 is fully illuminated by the X-ray beam, emanating from the X-ray tube 15, with the edge rays 25 and the middle ray 27. The measuring field 35 can likewise be scanned upon rotation of the second recording system, during which the X-ray tube 15 likewise moves on the circulating track 19. In the case of the present example embodiment, the detector 17 likewise has a number of rows of detector elements. However, only one detector row with detector elements 17a, 17b, . . . , etc. is shown in FIG. 2.

In the case of the present example embodiment having two recording systems with various detector sizes, the first recording system as a rule is the preferred recording system, which is also used without the second recording system to record projections of an object. However, when it is sensible it is also possible to use the second recording system without the first recording system. For example, if a moving organ such as the heart is to be examined at an increased data recording rate and with an increased temporal resolution, both recording systems are operated simultaneously, both recording systems preferably scanning the measuring field 35.

In order to be able to reconstruct informative images of an object with the aid of each of the recording systems of the computed tomography unit 1, it is necessary, as mentioned at the beginning, to determine for each recording system and before commissioning the computed tomography unit 1 for object measurements, to determine various correction tables for later signal processing, and to store the correction tables determined for the control and image computer 18 in a data memory 16 accessible to the control and image computer.

Specifically, the detector elements used to construct the two detectors of the two recording systems have manufacturing tolerances and therefore exhibit a measurement response differing slightly between them. The construction of the detectors of the recording systems from detector elements is performed, specifically, in such a way that the detector elements exhibit substantially the same behavior inside a detector. However, the detector elements of different detectors are not tuned to one another. For this reason, correction tables, for example, offset correction tables, channel error correction tables and radiation hardening correction tables are firstly obtained separately from one another for the two recording systems, and stored in the data memory 16 to be taken into account during later signal processing.

Water value scaling constitutes the last step in generating tables in the CT raw data processing. Even without water value scaling, it would be possible to reconstruct an image of an object simply with each of the two recording systems, since correction tables have already been determined for detector elements of the two detectors 13, 17 of the two recording systems. However, the CT values in an image reconstructed in such a way still have an offset that is to be removed by the water value scaling.

In water value scaling, a phantom 50 filled with water and which is, as a rule, a circular water disk of approximately 20 cm diameter is arranged in the opening 7 of the computed tomography unit 1 in such a way that the axis of rotation 9 and the central axis 51 of the water phantom are at least substantially aligned. Subsequently, the first recording system is firstly used while being rotated about the axis of rotation 9 to record projections at various projection angles of the water phantom 50, and an image of the water phantom 50 is reconstructed taking account of all previously determined correction values. Subsequently, a mean value M of the CT values of the image of the water phantom is formed from a centric, circular region of approximately 5 cm diameter. The water scaling factor is then determined by the equation
SKL(h, V)=1000:(1000+M).

The water scaling factor is a function of the slice thickness of the X-ray beam impinging on the detector 13, which can be set by way of the diaphragm 41 assigned to the X-ray tube 11. Moreover, the water scaling factor is a function of the high voltage applied to the X-ray tube 11. Consequently, various water scaling factors are determined for the first recording system as a function of the slice thickness h₁ and the applied tube high voltage V₁, and stored in the data memory 16.

In the same way, the second recording system is used as a function of the slice thickness h₂, which can be set by way of the diaphragm 45 assigned to the second X-ray tube 15, and as a function of the high voltage V₂ applied to the second X-ray tube, to determine several water scaling factors and store them in the data memory 16.

The two recording systems are then certainly set respectively per se. However, when measured values of the two recording systems are used in order to reconstruct an image of an examined object therefrom, data discontinuities that lead to artifacts in a reconstructed image result on the basis of the different scaling factors, based on the various correction tables, for the two recording systems. In order to counteract this, it is proposed to determine a further scaling factor for the measured values of the first or of the second recording system in order to at least reduce the artifacts.

In the case of the present example embodiment, the scaling factor for the measured values of the second recording system is determined. The first step in the course of determining the scaling factor is to record a projection of the water phantom 50 in the position on the first recording system illustrated in FIG. 2 (φ=0°) by way of the first recording system with a first slice thickness h₁ set by the diaphragm 41 and with a first voltage V₁ applied to the X-ray tube 11. For the sake of simplicity, only the detector rows of the detector 13 that are shown in FIG. 2 are considered below. The measured values of the detector elements of the detector 13 are buffered in this case in the data memory 16.

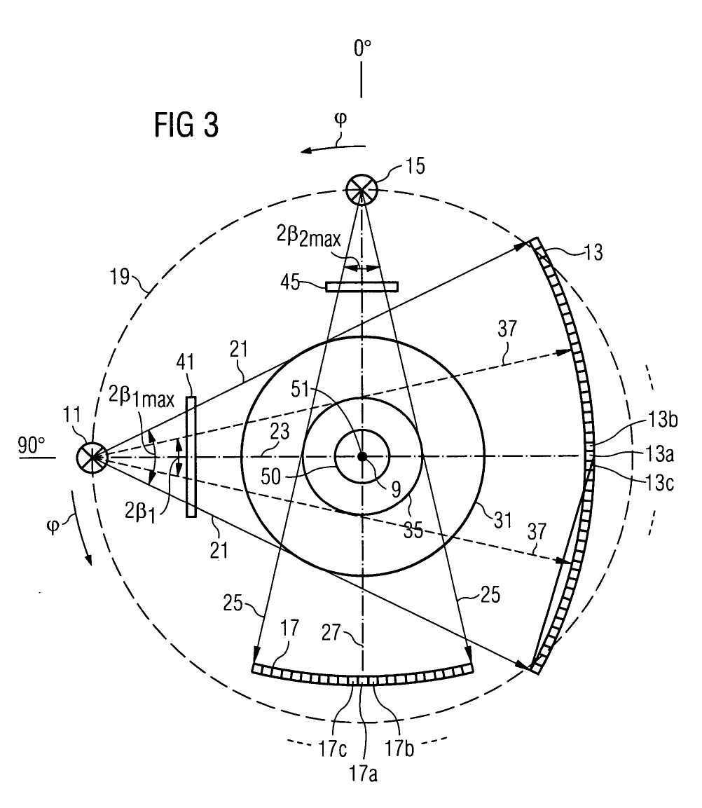

The two recording systems are then rotated by 90° in a counterclockwise sense (φ-direction) such that, as shown in FIG. 3, the detector 17 of the second recording system comes to lie at least substantially at the same spatial position as that previously of the detector 13 of the first recording system, that is to say the detector elements 13a and 17a or 13b and 17b or 13c and 17c etc. correspond to one another. The corresponding detector elements need not in this case occupy exactly the same spatial position. Rather, it suffices for their positions to correspond. This may be explained by way of example for the detector elements 13a and 17a.

After the 90° rotation, the spatial course of the central ray 27 corresponds at least substantially to the spatial course of the central beam 23 before the 90° rotation. The two detector elements 13a and 17a correspond to one another since the central ray 23 strikes the detector element 13a before the 900 rotation (FIG. 2), and the central ray 27 strikes the detector element 17a after the 90° rotation (FIG. 3). Mutually corresponding detector elements are thus struck by an X-ray beam in at least substantially the same spatial direction taking account of the 90° offset of the recording systems.

In this position, that is to say at φ=0° for the second recording system, a projection of the water phantom 50 is recorded as a function of the slice thickness h₂, set by the diaphragm 45 of the second recording system, which is equal to h₁, and as a function of the high voltage V₂, applied to the X-ray tube 15, which is equal to V₁, and the measured values of the detector 17 are buffered in the data memory 16. Thus, a projection of the water phantom 50 is therefore recorded by the two recording systems at the same projection angle (φ=0°), and a pair of projections of the two recording systems is thereby obtained. The detector elements of the two recording systems were located in this case substantially at the same spatial position or a mutually corresponding one during the recording of the respective projection. Consequently, corresponding pairs of detector elements or pairs of measured values can be formed, and the measured values of the pairs of detector elements can be compared with one another.

According to a first variant of the determination of a scaling factor, the measured values of corresponding detector elements, that is to say of detector elements that are located at least substantially at the same spatial position during recording of the respective projection, are divided, and averaging is carried out over the divided measured values. This computing operation is carried out with the aid of the control and image computer 18. Two projections, which form a pair of projections, already suffice to be able in this case to determine an associated scaling factor globally for the second recording system. This procedure is repeated for a number of different slice thicknesses h₁ and h₂ as well as for a number of different high voltages V₁ and V₂ applied to the X-ray tubes 11, 15, respectively. Global scaling factors are obtained in this way for the second recording system as a function, respectively, of the slice thickness and the high voltage applied to the X-ray tubes 11, 15, and are stored in the data memory 16. The scaling factors thus determined are available therefore to the control and image computer 18 for later reconstruction of images from recorded projections.

As a rule, however, it is not only that use is made only of one pair of projections in each case in order to determine the scaling factors as a function of slice thickness and tube voltage, but rather pairs of projections are respectively determined with the aid of the two recording systems for a pair of values composed of slice thickness and tube voltage at various projection angles. Such pairs of projections can be obtained in this case from one or more total rotations of the recording systems about the water phantom 50, or use is made of only pairs of projections from one or various segments of a scan of the water phantom 50 in order to determine the scaling factors.

In continuation of what has been described above, the measured values, belonging to a pair of projections, of corresponding detector elements are divided in this case and averaging is carried out over the divided measured values such that a mean value is present per pair of projections. In order to determine the associated scaling factor for the second recording system, averaging is subsequently carried out once more over the mean values of the pairs of projections. The scaling factors belonging to the various pairs of values composed of their thickness and tube voltage are stored in the data memory 16.

Alternatively, when use is made of a number of pairs of projections it is possible firstly to carry out averaging over the measured values of a detector element of each detector 13, 17 which originate from projections obtained at various projection angles. Subsequently, the averaged measured values of corresponding detector elements of the two detectors 13, 17 are divided, and averaging is carried out once more over the divided averaged measured values in order to determine a global scaling factor.

According to a further mode of procedure, averaging is firstly carried out for each pair of projections over the measured values of the detector elements of the respective projection, and the determined mean values of the projections are subsequently divided. Finally, so as to determine the scaling factor averaging is carried out again over the divided mean values given the use of a number of pairs of projections in order to obtain the associated scaling factor for the second recording system.

As already described, in the case of this procedure as well, scaling factors are determined for various slice thicknesses and for various high voltages applied to the X-ray tubes 11, 15, and stored in the data memory 16 in order to be able to scale the measured values of the second recording system with the aid of the scaling factor later in the case of reconstructions where use is made of measured values of both recording systems, such that the occurrence of artifacts is reduced or even completely avoided upon mixing of the measured values of the two recording systems during a reconstruction of an image.

The determination of the scaling factors for the second recording system, which is preferably performed as early as during the calibration of the recording systems of the computed tomography unit 1, can be carried out repeatedly during operation of the computed tomography unit 1, that is to say during object measurement, in order to be able to counter drift phenomena that can occur in the course of time. The determination of a scaling factor is performed here as in the case of the determination of a scaling factor with the aid of the water phantom 50.

In this case, two X-ray projections of an examination object, for example of the patient 5, that are recorded with the aid of the two recording systems at at least substantially the same projection angle in each case likewise form a pair of projections such that, as described above, the measured values can be compared with one another in the way described in order to determine a new scaling factor or to check the validity of an originally determined scaling factor and to correct the latter should drift phenomena have appeared. Precisely for this case, there is the option of using pairs of protections from one or various segments of a scan of the object, with the aim here necessarily being, in particular, to select projections whose measured values can be compared with one another effectively owing to the object properties.

The determination of the scaling factors is preferably performed for a setting of the recording systems in which both recording systems scan the measuring field 35. In this case, the aperture angles of the two recording systems are equal, and defined pairs of detector elements exist, as is to be gathered from FIGS. 2 and 3.

Embodiments of the invention were described above with reference to a computed tomography unit in which the second recording system has a smaller X-ray detector. However, the embodiments of invention can also be applied to computed tomography units with two recording systems whose X-ray detectors have the same size and extent.

Moreover, embodiments of the invention can also be applied to computed tomography units that include more than two recording systems. In this case, instead of pairs of projections it is necessary to form tuples of projections in order to be able in this case to determine scaling factors for the recording systems.

In the case of the present example embodiment, the scaling factors were determined for, or assigned to, the second recording system. However, the scaling factors can also be used for the first recording system by respectively using the reciprocal value of each scaling factor.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for determining at least one scaling factor for measured values obtained with the aid of a computed tomography unit, the method comprising:
determining a scaling factor for the measured values of at least one of two recording systems of a computed tomography unit on the basis of measured values that originate from projections recorded from an object with the aid of the two recording systems, wherein to determine the scaling factor, at least one projection at substantially the same projection angle is recorded with the aid of each of the two recording systems whose measured values are compared with one another.

2. The method as claimed in claim 1, wherein when determining the scaling factor measured values of corresponding detector elements of the two recording systems, which are located substantially at at least one of the same and a corresponding position in space during recording of the respective projection, are compared with one another in pairs.

3. The method as claimed in claim 2, wherein the measured values of corresponding detector elements of the two recording systems are divided.

4. The method as claimed in claim 3, wherein averaging is carried out over the divided measured values.

5. The method as claimed in claim 1, wherein averaging is carried out over the measured values of the respective projection of a recording system, and the mean values determined for the projections are divided.

6. The method as claimed in claim 1, wherein the determination of the scaling factor is performed with the aid of a number of projections of the two recording systems recorded at various projection angles.

7. The method as claimed in claim 1, wherein the determination of the scaling factor is performed with the aid of a number of recorded projections that are obtained in at least one sector given a circular rotation of the two recording systems.

8. The method as claimed in claim 1, wherein averaging is carried out over the measured values of a detector element of each detector that originate from projections obtained at various projection angles, and wherein the averaged measured values of corresponding detector elements of the two detectors are divided.

9. The method as claimed in claim 1, wherein the determination of the scaling factor is performed in the course of the water value scaling of the two recording systems before an object measurement.

10. The method as claimed in claim 9, wherein, during the water value scaling projections of a phantom provided with water are obtained at various projection angles with the aid of the first recording system, and a first water scaling factor is determined from the measured values of the first recording system in such a way that the CT values of the image produced by the phantom from the projections are on average at 0 HU.

11. The method as claimed in claim 9, wherein, during the water value scaling projections of a phantom provided with water are obtained at various projection angles with the aid of the second recording system, and a second water scaling factor is determined from the measured values of the second recording system in such a way that the CT values of the image produced by the phantom from the projections are on average at 0 HU.

12. The method as claimed in claim 1, wherein the determination of the scaling factor is performed during an object measurement with the aid of the two recording systems.

13. The method as claimed in claim 1, wherein a number of scaling factors are determined as a function of the slice thickness and the energy of the X-radiation.

14. The method as claimed in claim 1, wherein the two recording systems respectively include an X-ray tube, a number of scaling factors being determined as a function of the voltages applied to the X-ray tubes.

15. The method as claimed in claim 2, wherein the determination of the scaling factor is performed with the aid of a number of projections of the two recording systems recorded at various projection angles.

16. The method as claimed in claim 2, wherein the determination of the scaling factor is performed with the aid of a number of recorded projections that are obtained in at least one sector given a circular rotation of the two recording systems.

17. The method as claimed in claim 6, wherein averaging is carried out over the measured values of a detector element of each detector that originate from projections obtained at various projection angles, and wherein the averaged measured values of corresponding detector elements of the two detectors are divided.

18. The method as claimed in claim 7, wherein averaging is carried out over the measured values of a detector element of each detector that originate from projections obtained at various projection angles, and wherein the averaged measured values of corresponding detector elements of the two detectors are divided.

19. The method as claimed in claim 10, wherein, during the water value scaling projections of a phantom provided with water are obtained at various projection angles with the aid of the second recording system, and a second water scaling factor is determined from the measured values of the second recording system in such a way that the CT values of the image produced by the phantom from the projections are on average at 0 HU.

20. A computed tomography unit, comprising:
at least two recording systems, rotatable about a common rotation axis, and each including an X-ray source and a detector having detector elements for detecting X-radiation emanating from the X-ray source; and
means for determining a scaling factor for measured values of at least one of the two recording systems on the basis of measured values that originate from projections recorded from an object with the aid of the two recording systems, wherein to determine the scaling factor, at least one projection at substantially the same projection angle is recorded with the aid of each of the two recording systems whose measured values are compared with one another.
